(19) United States
(12) Patent Application Publication
(10) Pub. No.: US 2013/0096454 A1
(43) Pub. Date: Apr. 18, 2013

(54) IMAGE REPRODUCING APPARATUS AND METHOD FOR REPRODUCING REALTIME IMAGE THEREOF

(71) Applicants: Eun Hye JANG, Daejeon (KR); Sang Hyeob Kim, Daejeon (KR); Byoung-Jun Park, Iksan (KR); Myung-Ae Chung, Daejeon (KR)

(72) Inventors: Eun Hye JANG, Daejeon (KR); Sang Hyeob Kim, Daejeon (KR); Byoung-Jun Park, Iksan (KR); Myung-Ae Chung, Daejeon (KR)

(73) Assignee: Electronics and Telecommunications Research Institute, Daejeon (KR)

(21) Appl. No.: 13/529,941

(22) Filed: Jun. 21, 2012

(30) Foreign Application Priority Data
Oct. 17, 2011 (KR) 10-2011-0106044
Mar. 8, 2012 (KR) 10-2012-0023953

(51) Int. Cl. A61B 5/0484

(52) U.S. Cl. 600/544

(57) ABSTRACT

Disclosed is an image reproducing device which includes an image playback unit configured to play an image; a brain signal measuring unit configured to measure a brain signal from the human perceiving the played image; a brain signal analyzing unit configured to analyze the brain signal; a feature extracting unit configured to extract a feature from the analyzed brain signal; an image selecting unit configured to select images, corresponding to the feature from among the played images; and a brain image reproducing unit configured to obtain an average of the selected images to reproduce an image perceived through the brain.

### CROSS-REFERENCE TO RELATED APPLICATIONS

A claim for priority under 35 U.S.C. §119 is made to Korean Patent Application Nos. 10-2011-0106044 filed Oct. 17, 2011 and 10-2012-0023953 filed on Mar. 8, 2012, in the Korean Intellectual Property Office, the entire contents of which are hereby incorporated by reference.

### BACKGROUND

The inventive concepts described herein relate to a reproducing system, and more particularly, relate to an image reproducing device using a human cerebral signal and an image reproducing method thereof.

Sense of sight of the human may discriminate ten thousand colors, and focus of eyes of the human may shift by a unit of several centimeters within a wide range for 0.2 second. Further, the sense of sight of the human may identify objects in a dark place as well as in a light place, and may secure a sight of almost 180 degrees.

Now that it becomes an aging society thanks to the improved standards of living, there may increase cases that a function of sense of sight is lowered or lost due to blindness or illness causing visual impairment. There may have been researched artificial vision for sight recovery with advanced countries as the center.

The artificial vision may include cortical implant, optic nerve implant, retinal implant, bio-hybrid implant, chemical implant stimulating through neurotransmitter. The artificial vision may enable the human with the deteriorated faculty of sight to recover the vision to some degree.

However, the human may recognize objects by vision information input via eyes and the workings of the brain receiving the vision information. The brain of the human may simplify a much amount of vision information, and may make images by using and guessing vision information stored remaining at one's memory.

When vision information input via eyes does not match with information accepted by the brain, the human does not see objects properly. For example, this phenomenon may correspond to an optical illusion that arises due to a difference between vision information and information accepted by the brain. Since much vision information has been accumulated, the eyes of the human may function flawlessly. For this reason, a process of accumulating vision information of the brain may be needed for a normal function of eyes.

Besides information input via eyes, perception, visual image, dream, visual hallucination, and the like experienced by vision information of the brain may be very lifelike, and all things may continuously vary every moment. The variation may effect as strong and important experiences. There may be needed analysis through quantification of the brain based on a lifelike process for understanding of the vision function of the human.

### SUMMARY

One aspect of embodiments of the inventive concept is directed to provide an image reproducing device comprising an image playback unit configured to play an image; a brain signal measuring unit configured to measure a brain signal from the human perceiving the played image; a brain signal analyzing unit configured to analyze the brain signal; a feature extracting unit configured to extract a feature from the analyzed brain signal; an image selecting unit configured to select images, corresponding to the feature from among the played images; and a brain image reproducing unit configured to obtain an average of the selected images to reproduce an image perceived through the brain.

In example embodiments, the brain signal measuring unit measures a brain signal using at least one of fMRI (functional Magnetic Resonance Imaging), fNIR (functional Near Infra-Red), PET (Positron Emission Tomography), and EEG (Electroencephalography).

In example embodiments, the feature extracting unit extracts a feature using a difference between brain signals according to an image feature.

In example embodiments, the image reproducing device further comprises an image comparing and analyzing unit configured to compare and analyze the played image and the reproduced image.

Another aspect of embodiments of the inventive concept is directed to provide a method of reproducing an image, the method comprising playing an image; measuring a brain signal from the human perceiving the played image; analyzing the brain signal; extracting a feature from the analyzed brain signal; selecting images, corresponding to the feature from among the played images; and obtaining an average of the selected images to reproduce an image perceived through the brain.

In example embodiments, the method further comprises comparing and analyzing the played image and the reproduced image.

In example embodiments, the feature is extracted using a difference between brain signals according to an image feature.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a block diagram schematically illustrating an image reproducing device according to an embodiment of the inventive concept.

FIG. 2 is a flowchart illustrating an operation of an image reproducing device in FIG. 1.

FIG. 3 is a diagram illustrating an image reproducing operation according to an image reproducing device of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

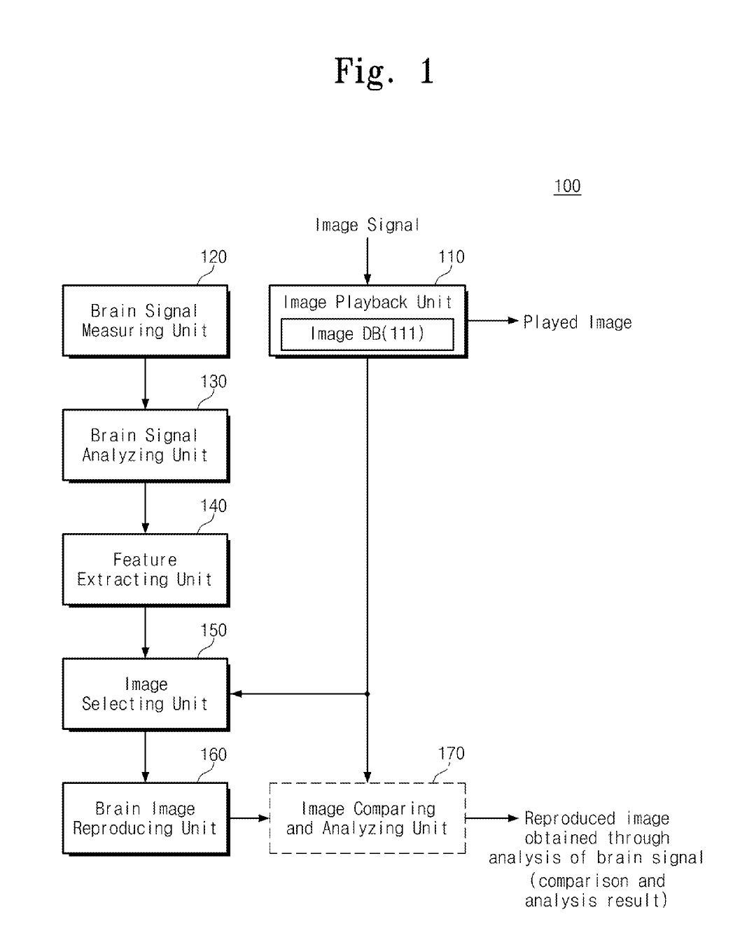

FIG. 1 is a block diagram schematically illustrating an image reproducing device according to an embodiment of the inventive concept.

Referring to FIG. 1, an image reproducing device 100 may include an image playback unit 110, a brain signal measuring unit 120, a brain signal analyzing unit 130, a feature extracting unit 140, an image selecting unit 150, and a brain image reproducing unit 160. The image reproducing device 100 may further include an image comparing and analyzing unit 170.

The image playback unit 110 may be configured to play an input image. The image playback unit 110 may include an image database 111 for storing image data. The image database 111 may store images to be played or images for image selection of the image selecting unit 150. The image database 111 can be placed outside the image playback unit 110.

The image playback unit 110 may be configured to play an image signal input from an external device or image data stored at the image database 111. The image playback unit 110 may store an input image signal at the image database 111. An image played by the image playback unit 110 may be provided to the human. The image playback unit 110 may provide the playback image to the image comparing and analyzing unit 170.

The brain signal measuring unit 120 may measure a brain signal of the human generated when the played image is recognized through eyes of the human. The brain signal measuring unit 120 may measure the brain signal using one of fMRI (functional Magnetic Resonance Imaging), fNIR (functional Near Infra-Red), PET (Positron Emission Tomography), and EEG (Electroencephalography). The brain signal measuring unit 120 may measure the working of the brain using the brain signal when the human sees the played image. The brain signal measuring unit 120 may output the measured brain signal to the brain signal analyzing unit 130.

The brain signal analyzing unit 130 may analyze the measured brain signal. The brain signal analyzing unit 130 may analyze the brain signal using a brain signal analyzing program. Herein, the brain signal analyzing unit 130 may analyze the brain signal to extract a feature from the brain signal. The brain signal analyzing unit 130 may output the analyzed brain signal to the feature extracting unit 140.

The feature extracting unit 140 may extract a feature from the analyzed brain signal. When the human sees a specific feature, the feature extracting unit 140 may extract a feature using a difference between brain signals according to the feature of the image. For example, the feature extracting unit 140 may check a difference between brain signals generated when the human sees sharp and round objects. The feature extracting unit 140 may extract a feature using a difference between brain signals. The feature extracting unit 140 may output the extracted feature to the image selecting unit 150.

The image selecting unit 150 may make comparison and analysis using the image database 111 that stores an image corresponding to the feature. The image selecting unit 150 may select at least one image having a response similar with the feature. Herein, the selected images may be images having the same brain response as the feature. The image selecting unit 150 may output the selected images to the brain image reproducing unit 160.

The brain image reproducing unit 160 may reproduce a brain image using an average of the selected images. The brain image reproducing unit 160 may output the reproduced image, that is, a reproduced image obtained through a brain signal to an external device. Also, the brain image reproducing unit 160 may output the reproduced image to the image comparing and analyzing unit 170.

The image playback unit 110 may include a display device which is configured to output the reproduced image of the brain image reproducing unit 160. Herein, the reproduced image may include vision information recognized by the brain.

The image comparing and analyzing unit 170 may compare and analyze a playback image of the image playback unit 110 and a reproduced image of the brain image reproducing unit 160. The image comparing and analyzing unit 170 may output a comparison and analysis result. The comparison and analysis result may indicate information associated with index of coincidence between the reproduced image and the playback image.

With the inventive concept, it is possible to reproduce an image perceived through the brain of the human using brain signals.

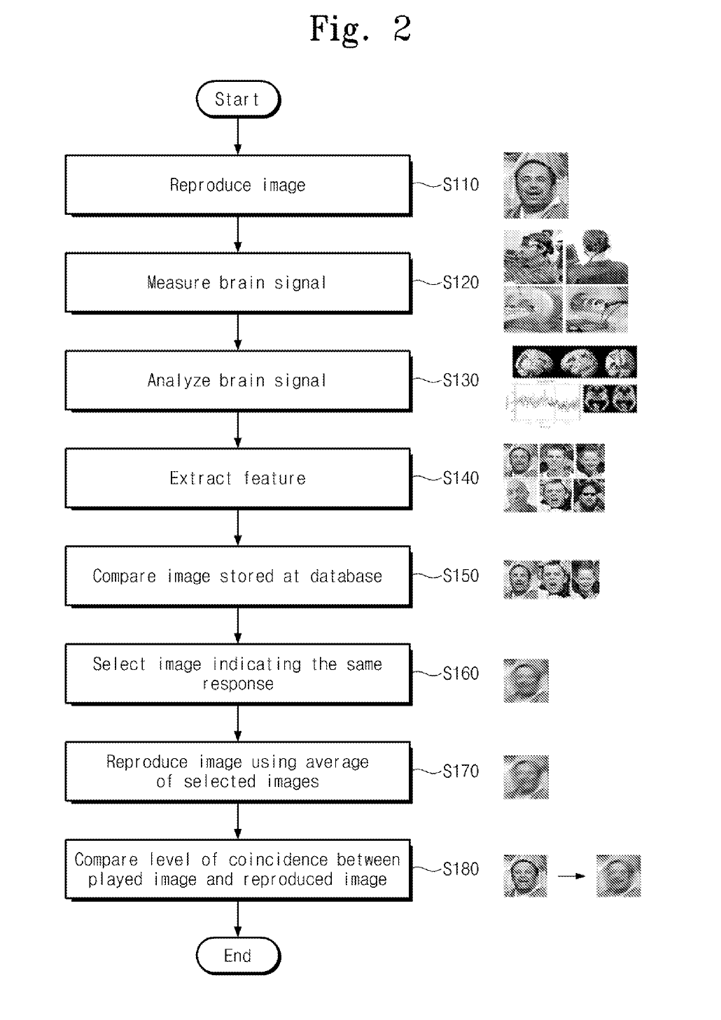

FIG. 2 is a flowchart illustrating an operation of an image reproducing device in FIG. 1.

In operation S110, an image playback unit 110 may play an input image. An image played by the image playback unit 110 may be provided to the human.

In operation S120, a brain signal measuring unit 120 may measure a brain signal when the human perceives the played image through eyes of the human. The brain signal measuring unit 120 may output the measured brain signal to a brain signal analyzing unit 130.

In operation S130, the brain signal analyzing unit 130 may analyze the measured brain signal. The brain signal analyzing unit 130 may output the analyzed brain signal to a feature extracting unit 140.

In operation S140, the feature extracting unit 140 may extract a feature from the analyzed brain signal. The feature extracting unit 140 may output the extracted feature to an image selecting unit 150.

In operation S150, the image selecting unit 150 may make comparison and analysis using an image database 111 that stores an image corresponding to the feature.

In operation S160, the image selecting unit 150 may select at least one image having a response similar to the feature. The image selecting unit 150 may output the selected images to a brain image reproducing unit 160.

In operation S170, the brain image reproducing unit 160 may reproduce a brain image using an average of the selected images.

An image comparing and analyzing unit 170 may compare and analyze a played image of the image playback unit 110 and a reproduced image of the brain image reproducing unit 160.

In FIG. 2, images or operations provided from modules are exemplarily illustrated at left sides of the operations.

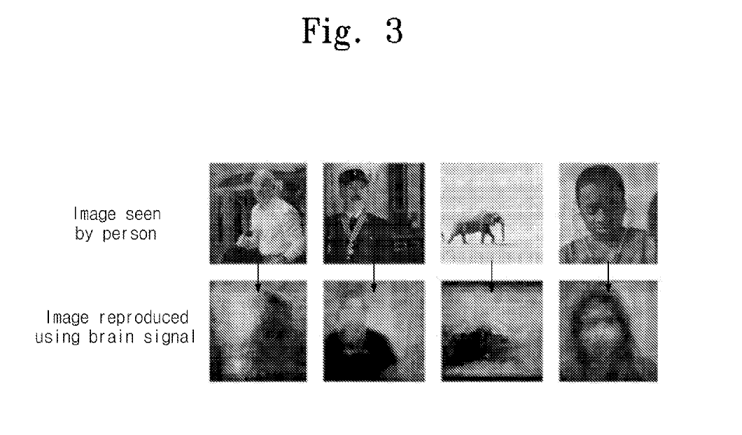

FIG. 3 is a diagram illustrating an image reproducing operation according to an image reproducing device of the inventive concept.

Referring to FIG. 3, there are illustrated an image reproduced by an image reproducing device of the inventive concept, that is, an image obtained through analysis of brain signals and an image seen by the human. As understood from FIG. 3, it is possible to confirm how the human perceives seen images through the brain.

With the inventive concept, it is possible to understand a vision function by analyzing the working of the brain based on a lifelike process through quantification. It may be applied to metical appliances or brain-machine interface devices for psychiatric diagnose through understanding of the vision function. Also, cataloged vision information of the brain may be applied to the intelligent robot technology as recognition and information input means.

Also, images seen through eyes of the human may be reproduced by analyzing brain signals. That is, it is possible to check vision information recognized by the brain and build a database. Thus, the inventive concept may be applied to sense assistance apparatuses for the blind and application equipment necessitating an artificial intelligence function such as a rehabilitation robot.

While the inventive concept has been described with reference to exemplary embodiments, it will be apparent to those skilled in the art that various changes and modifications may be made without departing from the spirit and scope of the present invention. Therefore, it should be understood that the above embodiments are not limiting, but illustrative.

What is claimed is:

1. An image reproducing device comprising:
an image playback unit configured to play an image;
a brain signal measuring unit configured to measure a brain signal from the human perceiving the played image;
a brain signal analyzing unit configured to analyze the brain signal;
a feature extracting unit configured to extract a feature from the analyzed brain signal;
an image selecting unit configured to select images, corresponding to the feature from among the played images; and
a brain image reproducing unit configured to obtain an average of the selected images to reproduce an image perceived through the brain.

2. The image reproducing device of claim 1, wherein the brain signal measuring unit measures a brain signal using at least one of fMRI (functional Magnetic Resonance Imaging), fNIR (functional Near Infra-Red), PET (Positron Emission Tomography), and EEG (Electroencephalography).

3. The image reproducing device of claim 1, wherein the feature extracting unit extracts a feature using a difference between brain signals according to an image feature.

4. The image reproducing device of claim 1, further comprising:
an image comparing and analyzing unit configured to compare and analyze the played image and the reproduced image.

5. A method of reproducing an image, comprising:
playing an image;
measuring a brain signal from the human perceiving the played image;
analyzing the brain signal;
extracting a feature from the analyzed brain signal;
selecting images, corresponding to the feature from among the played images; and
obtaining an average of the selected images to reproduce an image perceived through the brain.

6. The method of claim 5, further comprising:
comparing and analyzing the played image and the reproduced image.

7. The method of claim 5, wherein the feature is extracted using a difference between brain signals according to an image feature.
